(19) United States
(12) Patent Application Publication
(10) Pub. No.: US 2006/0008431 A1
(43) Pub. Date: Jan. 12, 2006

(54) Copolymer functionalized with an iodine atom, compositions comprising the copolymer and treatment processes

(76) Inventor: Celine Farcet, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/152,729

(22) Filed: Jun. 15, 2005

Related U.S. Application Data
(60) Provisional application No. 60/586,311, filed on Jul. 9, 2004.

(30) Foreign Application Priority Data
Jun. 15, 2004 (FR) 0451182

(51) Int. Cl. A61K 8/36

(52) U.S. Cl. 424/60

(57) ABSTRACT

The disclosure relates to copolymers functionalized with an iodine atom and including at least two different monomers. One of the at least two monomers is chosen from soluble monomers, and the other is chosen from insoluble monomers. The disclosure also relates to a cosmetic or dermatological composition comprising the copolymer in a physiologically acceptable medium. The disclosure also relates to a process for making up or caring for keratin materials using the composition.

This application claims benefit of U.S. Provisional Application No. 60/586,311, filed Jul. 9, 2004, the contents of which are incorporated herein by reference. This application also claims benefit of priority under 35 U.S.C. § 119 to French Patent Application No. 04 51182, filed Jun. 15, 2004, the contents of which are also incorporated by reference.

The present disclosure relates to novel copolymers and to compositions, such as cosmetic, pharmaceutical or dermatological topical compositions, including these copolymers.

In the field of cosmetics, compositions for obtaining a deposit, such as an adhesive or film-forming deposit, on keratin materials, such as the hair, the skin, the eyelashes or the nails, are often sought. For example, these compositions may provide color (i.e., in makeup or hair coloring compositions), sheen or a matt effect (i.e., in skincare or skin makeup compositions), physical properties such as shaping (i.e., in hair compositions, such as styling compositions), and protective or care properties (i.e., in care compositions, for example moisturizing or anti-UV compositions). Good remanance and good staying power of the cosmetic deposit over time, and also good adhesion to the support, are generally sought. Such deposits should be able, generally, to withstand mechanical attack such as rubbing or transfer on contact with another object; water, sweat, tears, rain, sebum and oils. This is particularly important for makeup, for example, lipsticks, where prolonged staying power of color and gloss, and transfer resistance of color are sought. This is also important for makeup such as foundations, eye shadows and face powders, where staying power of the color supplied by the composition is sought, while simultaneously maintaining the transfer resistance and also the matt effect exhibited by the initial shade for as long as possible, despite the secretion of sebum and sweat by the wearer. In addition, makeup compositions that are comfortable to wear and do not have an excessively tacky texture are desired.

The above-recited properties often conflict with one another. To achieve a composition that reconciles these properties within the same composition, a mixture of several different polymers, of different chemical nature, is generally used, with each polymer providing at least one of the desired characteristics. However, polymers, having different chemical natures, within such a mixture are not necessarily compatible with one another. This may result in the polymer mixture exhibiting demixing problems within the composition.

It is known to add random polymers, such as conventional acrylic polymers obtained by standard free-radical polymerization via random mixing of monomers, to a composition containing a mixture of polymers, in an attempt to solve the demixing problem. However, these random polymers are known to exhibit dispersity in the polymer chains of the composition, which itself can lead to demixing. Thus, the addition of these random polymers does not solve this problem satisfactorily.

In addition, the process of atom transfer radical polymerization (ATRP) is known and described in U.S. Pat. No. 5,807,937. ATRP allows the preparation of certain polymers that may be functionalized at their end, for example, with a bromine or chlorine atom. However, this process requires the use of metal catalysts, trace amounts of which may remain in the polymers thus prepared. If these polymers are used for cosmetic applications, the presence of trace amounts metal catalyst in the polymer may pose a problem. In addition, Br and Cl atoms are not always labile enough to be easily replaced with more desired functions.

Further, polymers that are functionalized at their end and are obtained by degenerative transfer (DT) are known and described in document No. WO99/20659. Free-radical polymerization controlled via DT allows the synthesis of functionalized (co)polymers of controlled molar mass and architecture. However, most polymers that have been prepared via this technique are homopolymers.

The present disclosure relates to novel copolymers that are cosmetically acceptable and functionalized on at least one of their ends with an atom that is labile enough to be readily replaced.

Further, copolymers of the present disclosure may avoid the prior art problems of demixing within the formula while at the same time providing desired cosmetic properties.

One aspect of the present disclosure is a copolymer functionalized on at least one of its ends with an iodine atom, and comprising at least two different monomers. One of the monomers of the at least two different monomers is chosen from soluble monomers, and the other of the at least two different monomers is chosen from insoluble monomers, as defined below.

Another aspect of the disclosure is a copolymer that may be obtained via free-radical polymerization controlled by degenerative transfer at the iodine atom:
of at least one free-radical initiator,
of at least one transfer agent comprising at least one iodine atom, and
of at least two different monomers, wherein one is chosen from soluble monomers and the other from insoluble monomers, as defined below.

Another aspect of the disclosure is a cosmetic or dermatological composition comprising, in a physiologically acceptable medium, at least one copolymer as defined above.

Copolymers according to the present disclosure may be easy to use in organic cosmetic media, such as solvent-based media and/or cosmetic oil media, while at the same time retaining advantageous rheological properties.

Further, the copolymers according to the disclosure exhibit good liposolubility in cosmetic solvents and/or cosmetic oils.

For the purpose of the present disclosure, the term, “soluble polymer” means a polymer that does not form a precipitate when dispersed in a solvent. In one embodiment of the present disclosure, the copolymer is soluble at a concentration of at least 3% by weight in isododecane at 25° C. and 1 atm, such as at least 5% or at least 10% by weight.

The copolymers of the present disclosure may be in various forms, such as in the form of a block or gradient polymer. In one embodiment, the copolymer of the present disclosure is in the form of a gradient polymer. In this embodiment, the copolymer may, for example, be utilized in high concentrations in the cosmetic compositions of the disclosure.

In a gradient copolymer, the composition within the polymer chain follows a composition gradient. In one embodiment of the disclosure, the gradient copolymer has a low composition polydispersity, meaning that all of the copolymer chains have an approximately analogous composition (i.e. sequence of monomers) and are therefore of homogeneous composition. As a result, cosmetic compositions comprising these copolymers do not exhibit the drawbacks and/or limitations of the compositions of the prior art.

The copolymer according to the disclosure may be linear gradient copolymer, represented by the formula:
F-[M₁M₂]_{gradient}-I
in which F represents a residue derived from the transfer agent (the radical R defined below) or from the initiator; I is an iodine atom; and M₁ and M₂ are monomers or a mixture of monomers.

According to one embodiment, the copolymers according to the disclosure may also be in block polymer form. For example, the polymers of the present disclosure may be in the form of a polymer comprising at least two different successive blocks, such as two successive blocks of different chemical nature. Each block of the copolymer according to the disclosure may be derived from one or more types of monomer. Therefore each block may consist of a homopolymer or a copolymer. If a block is a copolymer, this copolymer may be a random, alternating or gradient copolymer. As a result, the monomer distribution within each block may be random or controlled, depending on the nature and/or reactivity of the monomers.

If the copolymer of the present disclosure is a block copolymer, it may be a diblock polymer of the AB type; or a triblock polymer, i.e. of the type ABA, BAB or ABC, wherein C is different from A and B; or multiblock polymers containing more than three blocks, i.e. of the type (AB)n, (ABA)n, (BAB)n, (ABC)n or ABCD, wherein A, B, C and D are of different chemical nature. In one embodiment of the present disclosure, the copolymer comprises at least 2 or 3 successive blocks, wherein the two successive blocks are different. For example, the copolymer may be of the AB, ABA or ABC type.

If the copolymer according to the present disclosure is a linear block copolymer, it may be represented schematically by the formula: F-[(M₁)ₙ-(M₂)ₘ]I, in which n and m are integers greater than 1.

According to one embodiment of the disclosure, the copolymers according to the disclosure may also be in the form of a star polymer, wherein each branch of the star may be in gradient or block form. Star polymers are obtained when the transfer agent utilized is polyfunctional and includes at least three iodine atoms.

If the copolymer is a a star copolymer, it may be represented by the formula F-[M₁M₂-I]ₙ, wherein: M₁ and M₂ are different and represent a single monomer or a monomer mixture, and are arranged in a gradient (F-[(M₁M₂)_{grad}-I]_{n′}) or block (F-[(M₁)ₙ(M₂)ₘ-I]_{n′}) form; and n′ represents the number of branches in the star, and is an integer greater than 2, such as from 3 to 8.

If n′ is equal to 2, the polymer obtained is linear and difunctional.

According to yet another embodiment, the copolymer according to the present disclosure may be a grafted copolymer, the skeleton of which may be in gradient or block form. Grafted polymers may be obtained when one or more of the monomers M₁ and/or M₂ is a macromonomer as defined below.

According to the present disclosure, the copolymers may be in block, gradient, linear-chain, grafted or star form.

In one embodiment, the copolymer is a linear, gradient or block copolymer.

The number-average molecular mass of the copolymer according to one embodiment of the present disclosure may be from 2000 g/mol to 1,000,000 g/mol, such as from 3000 g/mol to 500,000 g/mol, from 4000 g/mol to 200,000 g/mol, or from 5000 g/mol to 50,000 g/mol.

As used in the present disclosure, the number-average molecular mass (Mn) is determined via liquid gel permeation chromatography (GPC) using a THF eluent and a refractometric detector. Calibration is performed using linear polystyrene standards.

The copolymers of the present disclosure may be obtained by free-radical polymerization controlled by degenerative transfer at the iodine atom. For purposes of this disclosure, the term, “controlled free-radical polymerization” denotes polymerization for which the side reactions that usually lead to the termination or transfer of the the propagating species are rendered highly unlikely, relative to propagation reactions carried out by means of a free-radical control agent. However, when the concentration of free radicals become high relative to the monomer concentration, these side reactions become determining factors and generally result in a polymer having a broader mass distribution.

During controlled free-radical polymerization, the polymer chains of the gradient copolymers of the disclosure grow simultaneously, and thus incorporate the same ratio of comonomers over time. Therefore, all of the chains have the same or similar structure, resulting in low composition dispersity. Further, these chains also have a low mass polydispersity index. As mentioned above, gradient copolymers are copolymers having a concentration gradient of the various monomers along the chain. The distribution of the polymer chains of the comonomers depends on the relative concentrations of the comonomers during the synthesis. The gradient copolymers according to the disclosure comprise at least two different monomers, the concentration of which changes gradually along the polymer chain in both a systematic and predictable manner. Therefore, all of the polymer chains have at least one monomer Mi for which, irrespective of the normalized position x on the polymer chain, there is a non-zero probability of finding this same monomer along the chain. One of the defining characteristics of a gradient copolymer is the fact that at any instant in the polymerization, all of the chains are subjected to the presence of all of the monomers. Thus, in the reaction medium, the concentration of each monomer is always non-zero, at any instant in the polymerization.

This fact makes it possible to differentiate between gradient copolymers and block polymers in which the evolution of the monomers along the polymer chain is not systematic: for example, for a diblock copolymer AB, within the sequence A, the concentration of the monomer B is always zero.

In the case of random polymers, the concentration gradient of the monomers along the polymer chain will not be gradual, systematic and predictable.

Moreover, among gradient copolymers in general, copolymers with a natural gradient and copolymers with an artificial gradient may be distinguished.

A copolymer with a natural gradient is a gradient copolymer batch-synthesized starting with an initial mixture of the comonomers. The distribution in the chain of the various monomers follows a law deduced from the relative reactivity and the initial monomer concentrations. These copolymers constitute the simplest class of gradient copolymers, because the initial mixture defines the final property of the product.

A copolymer with an artificial gradient is a copolymer whose monomer concentration is varied during the synthesis. In this case, the relative concentration of monomers in the chain of the copolymer changes due to a sudden and abrupt change of the monomers and/or monomer concentration in the reaction medium. Further, one or more monomers may disappear, to the benefit of one or more others.

Moreover, in a gradient copolymer, the relative distribution of the compositions between the various chains of the copolymer is narrow. In particular, there is no overlap between the peak for the gradient copolymer and those for the respective homopolymers. This means that the material obtained by gradient polymerization includes polymer chains having the same composition, whereas by standard random polymerization, various kinds of chain coexist, including those of the respective homopolymers.

Factors which impact the gradient include the relative reactivity coefficients of each monomer (known as rᵢ for the monomer Mi). These reactivity coefficients depend primarily on the type of synthetic process (homogeneous or dispersed) and solvents used. However, these coefficients are also impacted by the initial concentrations of each of the monomers, and by changes in monomer concentration, i.e. through the addition of monomers during the polymerization.

In a non-limiting embodiment of the present disclosure, the copolymers are prepared according to a degenerative transfer (DT) or a degenerative iodine transfer (DIT) process. These processes allow the formation of copolymers that are functionalized (i.e. mono- or multi-functionalized), on at least one of their ends, with an iodine atom. This process is described in patent application No. WO 99/20659.

In the DT or DIT processes, the chosen monomers are reacted with at least one polymerization initiator in the presence of an iodine-containing transfer agent.

The at least one polymerization initiator may be chosen from any initiator known to those skilled in the art for use in free-radical polymerization processes. Non-limiting examples of such initiators include azo type compounds, such as azobisisobutyronitrile; peroxide type compounds, such as organic hydroperoxides or peroxides containing 6-30 carbon atoms, for example benzoyl or didecanoyl peroxide; as well as redox couples, peresters, percarbonates or persulfates.

In a non-limiting embodiment of the present disclosure, the at least one initiator is chosen from organic peroxides containing from 8 to 30 carbon atoms, such as the didecanoyl peroxide sold under the reference PERKADOX® SE-10 by the company Akzo Nobel.

Further non-limiting examples of the at least one initiator include 2,5-bis(2-ethylhexanoylperoxy)-2,5-dimethylhexane (TRIGONOX 141 from the company Akzo Nobel) and tert-butyl peroxy-2-ethylhexanoate (TRIGONOX 21S from the company Akzo Nobel).

The at least one iodine containing transfer agent may be represented by the formula R-I, in which R is a linear, branched or cyclic, saturated or unsaturated alkyl radical containing from 1 to 30 carbon atoms. R may optionally comprise 1 to 4 additional iodine atoms, one or more fluorine atoms, and/or one or more functional groups chosen from CN and COOH.

In a non-limiting embodiment of the present disclosure, R is chosen from alkyls containing 1 to 6 carbon atoms, and may comprise one or more halogen atoms, such as fluorine, and/or a CN function. For example, R may be a perfluoro C1-C6 alkyl or a C1-C6 alkyl bearing a CN function.

Non-limiting examples of the at least one transfer agent that may be mentioned include iodo-1-perfluorohexane, iodoacetonitrile (ICH₂—CN), iodo-1-methane, diiodomethane, iodoform or triiodomethane, iodo-1-perfluoroisopropane, diiodoperfluorohexane, iodo-1-phenylethane, iodo-1-propane, iodo-1-isopropane, iodo-1-phenyl, 1,4-diiodophenyl and iodo-1-tert-butane.

Further, the at least one transfer agent may be macromolecular and may be in the form of a polymer, homopolymer or copolymer, obtained via a prior step of degenerative iodine transfer polymerization, and thus functionalized with at least one iodine atom.

According to one embodiment of the disclosure, transfer agents prepared by DIT are suitably used to prepare block copolymers.

According to one embodiment, the molar proportion r between the transfer agent and the initiator may range from 0.1 to 20, such as from 1 to 10, or from 2 to 5.

The molar proportion DP between the set of monomers and the transfer agent may be greater than 10, and may range from 50 to 1000, for example, from 100 to 500.

The copolymers of the present disclosure may be prepared, for example, by a person skilled in the art according to the following procedure:

1) A mixture of the various monomers is prepared, optionally in a solvent, preferably in a reactor, and is stirred. A free-radical polymerization initiator and a transfer agent are added. The mixture is preferably under an atmosphere of inert gas relative to a free-radical polymerization, such as nitrogen or argon.

The optional polymerization solvent may be chosen from cosmetic solvents and/or oils as defined below. For example, the polymerization solvent may be chosen from alkyl acetates such as butyl acetate or ethyl acetate, aromatic solvents such as toluene or alkanes such as isododecane, heptane or isohexadecane.

In general, a polymerization solvent in which the monomers and the resulting polymer are soluble is chosen.

2) The mixture is brought to the desired polymerization temperature and is stirred. The polymerization temperature may, for example, be within a range from 20° C. to 120° C., such as from 40° C. to 90° C.

The choice of the polymerization temperature may be optimized as a function of the chemical composition of the monomer mixture.

3) The polymerization medium is optionally modified during the polymerization, before reaching 90% conversion of the initial monomers, by supplemental addition of one or more monomers, such as a monomer from the initial mixture, or the same monomer combination as in the initial mixture. This addition may be performed in various ways, including abrupt addition of a single portion of additional monomer(s), or via continuous addition of monomer(s) over the time period during which polymerization takes place.

4) The polymerization reaction terminates when the desired degree of conversion is reached. The overall composition of the copolymer depends on the amount of conversion. In a non-limiting embodiment of the present disclosure, the polymerization is terminated after having reached at least 50% conversion. In other non-limiting embodiments, the polymerization reaction is terminated after reaching at least 60% or at least 90% conversion.

5) After the polymerization reaction is terminated, residual monomers which may remain can be removed by known methods, such as by evaporation, or by addition of an amount of standard polymerization initiator such as peroxide or azo derivatives.

6) If a block copolymer is desired, the additional monomer(s), and optionally the transfer agent and the initiator, may be added in an additional step, to form a second block.

The copolymer of the present disclosure may be obtained by free-radical polymerization either in bulk or in solution in an organic medium. The monomers may be added simultaneously, batch-wise, semi-continuously or consecutively.

For batch polymerization, the optional solvent, the monomers and the initiator may be mixed together in a reactor and heated to the required temperature.

For semi-continuous polymerization, all or some of the optional solvent, some of the monomers, i.e. from 1% to 20% by weight relative to the total weight of monomer, some of the initiator, i.e. from 1% to 20% by weight relative to the total weight of initiator, and optionally some of the transfer agent, i.e. from 0.1% to 20% by weight relative to the total weight of transfer agent, are introduced into a reactor and the mixture is heated to the required temperature. The remaining solvent of the monomers and the initiator is introduced by flow addition during the polymerization. The remaining constituents may then be introduced via identical or different, simultaneous or separate flow additions.

In a non-limiting embodiment of the present disclosure, a block copolymer is formed by a process in which the first block is formed by polymerization of the first monomer or mixture of first monomers, after which the the second monomer or mixture of monomers is added batch-wise or semi-continuously.

In a non-limiting embodiment of the present disclosure, a copolymer having a composition gradient is prepared via a process where the monomers are introduced batch-wise or semi-continuously. The polymerization of the first monomer, or mixture of first monomers, is started, and the second monomer, or mixture of second monomers, is added simultaneously, batch-wise, or semi-continuously, before the polymerization of the first monomer is complete.

Finally, according to one embodiment, the copolymer of the present disclosure comprises at least two different monomers, wherein at least one is chosen from “soluble” monomers, and the other is chosen from “insoluble” monomers.

For the purpose of the present disclosure, the term “soluble monomer,” means any monomer whose homopolymer is soluble, i.e. does not form a precipitate in the solvent, at a concentration of at least 3% by weight in isododecane at 25° C. and 1 atm.

In various non-limiting embodiments of the present disclosure, the at least one soluble monomer is chosen from monomers whose homopolymer is soluble at a concentration of at least 5% by weight, for example, 10% by weight, in isododecane at 25° C. and 1 atm.

For the purpose of the present disclosure, the term “insoluble monomer,” means any monomer whose homopolymer is insoluble at a concentration of at least 3% by weight in isododecane at 25° C. and 1 atm.

In various non-limiting embodiments of the present disclosure, the soluble monomers may be from the following monomers:
(i) (meth)acrylates of formula CH₂═CHCOOR or CH₂═C(CH₃)COOR in which R represents:
   a linear or branched alkyl group containing 8 to 30 carbon atoms, wherein the alkyl group is optionally: a) intercalated with one or more hetero atoms chosen from O, N, S and P and/or; b) substituted with one or more substituents chosen from —OH, halogen atoms (i.e. Cl, Br, I and F), and groups —NR4R5, in which R4 and R5 may be identical or different and represent hydrogen or a linear or branched C1 to C6 alkyl group or a phenyl group;
   a C8 to C12 cycloalkyl group, Non-limiting examples of R include octyl, decyl, lauryl, isooctyl, isodecyl, dodecyl, t-butylcyclohexyl, stearyl, 2-ethylhexyl, isobornyl, and behenyl groups;
(ii) tert-butyl methacrylate and isobutyl acrylate;
(iii) (meth)acrylamides of the formula CH₂═CHCONR₄R₅ or CH₂═C(CH₃)CONR₄R₅ in which:
   R4 represents a hydrogen atom or a linear or branched alkyl group containing from 1 to 6 carbon atoms; preferably, R4 is chosen from: hydrogen, methyl, ethyl, propyl, isopropyl, n-butyl, isobutyl, tert-butyl, hexyl, isohexyl, cyclohexyl;
   R5 represents a linear or branched alkyl group containing from 8 to 18 carbon atoms, such as an octyl, isooctyl, decyl, isodecyl, cyclodecyl, dodecyl, cyclododecyl, isononyl, undecyl, lauryl, t-butylcyclohexyl, stearyl, or 2-ethylhexyl group;
(iv) vinyl ethers of formula R₆O—CH═CH₂, or vinyl esters of formula: R₆—COO—CH═CH₂, in which R₆ represents a linear or branched alkyl group containing from 8 to 22 carbon atoms; the salts thereof; and mixtures thereof.

The macromonomer may be any polymer, such as an oligomer, comprising on only one of its ends an end group, i.e., a polymerizable end group, capable of reacting during the polymerization reaction with the monomers under consideration, to form the side chains of the polymer; this end group may be an ethylenically unsaturated group capable of undergoing free-radical polymerization with the monomers constituting the skeleton. The macromonomer allows the side chains of the copolymer to be formed. The polymerizable group of the macromonomer may advantageously be an ethylenically unsaturated group capable of undergoing free-radical polymerization. The said polymerizable end group may be in particular a vinyl or (meth)acrylate group.

Non-limiting examples of suitable macromonomers that may be used include the following compounds:
(v)a) linear or branched C8-C22 alkyl (meth)acrylate homopolymers and copolymers containing a polymerizable end group chosen from vinyl or (meth)acrylate groups, including but not limited to poly(2-ethylhexyl acrylate) macromonomers containing a mono(meth)acrylate end; poly(dodecyl acrylate) or poly(dodecyl methacrylate) macromonomers containing a mono(meth)acrylate end; and poly(stearyl acrylate) or poly(stearyl methacrylate) macromonomers containing a mono(meth)acrylate end. Such macromonomers are described in patents EP 895 467 and EP 96459.
(v)b) polyolefins containing an ethylenically unsaturated end group, including but not limited to those containing a (meth)acrylate end group. Non-limiting examples of such polyolefins include the following macromonomers, it being understood that they have a (meth)acrylate end group: polyethylene macromonomers, polypropylene macromonomers, polyethylene/polypropylene copolymer macromonomers, polyethylene/polybutylene copolymer macromonomers, polyisobutylene macromonomers; polybutadiene macromonomers; polyisoprene macromonomers; polybutadiene macromonomers; and poly(ethylene/butylene)-polyisoprene macromonomers. Such macromonomers are described in U.S. Pat. No. 5,626,005, which discloses ethylene/butylene and ethylene/propylene macromonomers containing a (meth)acrylate reactive end group. Non-limiting mention is also made of poly(ethylene/butylene) methacrylate, such as the product sold under the name KRATON LIQUID L-1253 by Kraton Polymers.
(vi) ethylenic hydrocarbons containing from 2 to 10 carbons, including but not limited to ethylene, isoprene or butadiene.

In a non-limiting embodiment of the present disclosure, the at least one soluble monomer is chosen from:
(meth)acrylates of formula CH₂═CHCOOR or CH₂═C(CH₃)COOR, in which R represents a linear or branched alkyl group containing 8 to 30 carbon atoms or a C8 to C12 cycloalkyl group;
linear or branched C8-C22 alkyl (meth)acrylate homopolymers and copolymers containing a polymerizable end group chosen from vinyl or (meth)acrylate groups containing at least one polymerizable end group;
polyolefins containing an ethylenically unsaturated end group, including those containing a (meth)acrylate end group.

Non-limiting examples of soluble monomers that may be utilized include isobornyl acrylate, isobornyl methacrylate, 2-ethylhexyl acrylate, 2-ethylhexyl methacrylate, lauryl acrylate, lauryl methacrylate, stearyl acrylate, stearyl methacrylate, tert-butyl methacrylate, isobutyl acrylate; behenyl methacrylate, behenyl acrylate; and also poly(2-ethylhexyl acrylate) macromonomers containing a mono(meth)acrylate end group; poly(dodecyl acrylate) or poly(dodecyl methacrylate) macromonomers containing a mono(meth)acrylate end group; polystearyl (meth)acrylate macromonomers containing a mono(meth)acrylate end group; as well as ethylene/butylene and ethylene/propylene macromonomers containing a (meth)acrylate reactive end group.

In a non-limiting embodiment of the present disclosure, the at least one insoluble monomer may be chosen from the following monomers:
(i) (meth)acrylates of formula CH₂═CHCOOR or CH₂═C(CH₃)COOR in which R represents:
with the exclusion of tert-butyl methacrylate and isobutyl acrylate, a linear or branched alkyl group containing 1 to 6 carbon atoms, in which the alkyl group is optionally: a) intercalated with one or more hetero atoms chosen from O, N, S and P; or b) substituted with one or more substituents chosen from —OH, halogen atoms (Cl, Br, I and F), groups —NR₄R₅, in which R₄ and R₅ may be identical or different, and represent hydrogen or a linear or branched C1 to C6 alkyl phenyl group, a polyoxyalkylene group having 5 to 30 repeating polyoxyalkylene groups, such as polyoxyethylene and/or polyoxypropylene;
a C3 to C6 cycloalkyl group, wherein the cycloalkyl group optionally comprises one or more hetero atoms chosen from O, N, S and/or P, and/or may be substituted with one or more substituents chosen from —OH and halogen atoms (such as Cl, Br, I and F);
a C5 to C8 aryl or C6 to C10 aralkyl group (i.e. an aryl group having a C1 to C5 alkyl group); wherein, in a non-limiting embodiment, R may be chosen from a methyl, ethyl, propyl, isopropyl, n-butyl, isobutyl, tert-butyl, hexyl, cyclohexyl, 2-hydroxyethyl, 2-hydroxybutyl, 2-hydroxypropyl, methoxyethyl, ethoxyethyl, methoxypropyl, phenyl, 2-phenylethyl, t-butylbenzyl, benzyl, furfurylmethyl or tetrahydrofurfurylmethyl, methoxypolyoxyethylene (or POE-methyl) group; POE-behenyl, trifluoroethyl; dimethylaminoethyl, diethylaminoethyl, or dimethylaminopropyl group;
(ii) (meth)acrylamides of the formula CH₂═CHCONR₄R₅ or CH₂═C(CH₃)CONR₄R₅, in which R4 and R5 may be identical or different and represent a hydrogen atom or a linear or branched alkyl group containing from 1 to 6 carbon atoms, wherein said linear or branched alkyl group is optionally: a) intercalated with one or more hetero atoms chosen from O, N, S and P; or b) substituted with one or more substituents chosen from hydroxyl groups, halogen atoms (i.e. Cl, Br, I and F) and groups —NR′₄R′₅, in which R′ and R′5 may be identical or different, and represent a C1 to C4 alkyl group; in a non-limiting alternative embodiment, R₄ represents a hydrogen atom and R₅ represents a 1,1-dimethyl-3-oxobutyl group; Non-limiting examples of suitable R4 and R5 groups include: hydrogen, methyl, ethyl, propyl, ispropyl, n-butyl, isobutyl, tert-butyl, hexyl, isohexyl, cyclohexyl, dimethylaminoethyl, diethylaminoethyl, and dimethylaminopropyl;
(iii) ethylenically unsaturated monomers comprising at least one carboxylic, phosphoric or sulfonic acid or anhydride function, for instance acrylic acid and methacrylic acid; crotonic acid, maleic anhydride, itaconic acid, fumaric acid, maleic acid, styrenesulfonic acid, vinylbenzoic acid, vinylphosphoric acid, acrylamidopropanesulfonic acid, and the salts thereof;
(iv) the vinyl ethers of formula R₆O—CH═CH₂ or the vinyl esters of formula R₆—COO—CH═CH₂ in which R₆ represents a linear or branched alkyl group containing from 1 to 6 atoms;
(v) styrene and its derivatives, such as methylstyrene, chlorostyrene or chloromethylstyrene;
(vi) silicone macromonomers, such as polydimethylsiloxane, containing a mono(meth)acrylate end group, such as those of formula (IIa) below: 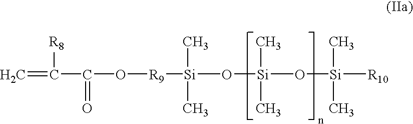
   in which:
R₈ denotes a hydrogen atom or a methyl group; in a non-limiting embodiment R₈ is methyl;
R₉ denotes a linear or branched, divalent hydrocarbon-based group containing from 1 to 10 carbon atoms and optionally includes one or two ether bonds —O—; i.e., ethylene, propylene or butylene; in a non-limiting embodiment R₉ is linear;
R₁₀ denotes a linear or branched alkyl group containing from 1 to 10 carbon atoms, i.e., from 2 to 8 carbon atoms; In a non-limiting embodiment, R₁₀ is methyl, ethyl, propyl, butyl or pentyl;
n denotes an integer ranging from 1 to 300, i.e., from 3 to 200 or from 5 to 100.

Non-limiting examples of silicone macromonomers include monomethacryloyloxypropyl polydimethylsiloxanes, such as those sold under the name PS560-K6 by UCT (United Chemical Technologies Inc.) or under the name MCR-M17 by Gelest Inc;
(vii) ethylenically unsaturated monomers comprising at least one tertiary amine function, such as 2-vinylpyridine or 4-vinylpyridine;
(viii) vinyl compounds of formula: CH2═CH—R₉, CH2═CH—CH2-R₉ or CH2═CH(CH3)-CH2-R₉
   in which R₉ is a hydroxyl group, a halogen (i.e. Cl or F), NH2, OR₁₀ in which R₁₀ is a phenyl group or a C1 to C12 alkyl group (i.e. the monomer is a vinyl or allylic ether);
(ix) ethylenically unsaturated monomers comprising one or more silicon atoms, such as methacryloyloxypropyltrimethoxysilane and methacryloyloxypropyltris(trimethylsiloxy)silane;
(x) perfluorooctyl acrylate.

In a non-limiting embodiment of the present disclosure, the at least one insoluble monomer is chosen from:
(meth)acrylates of formula CH₂═CHCOOR or CH₂═C(CH₃)COOR, in which R represents, with the exclusion of tert-butyl methacrylate and isobutyl acrylate, a linear or branched alkyl group containing 1 to 6 carbon atoms or a C3 to C6 cycloalkyl group;
(meth)acrylamides of formula CH₂═CHCONR₄R₅ or CH₂═C(CH₃)CONR₄R₅,
   in which R₄ and R₅ may be identical or different, and represent a hydrogen atom or a linear or branched alkyl group containing from 1 to 6 carbon atoms; In a more specific non-limiting embodiment of the present disclosure, R₄ represents a hydrogen atom and R₅ represents a 1,1-dimethyl-3-oxobutyl group;
ethylenically unsaturated monomers comprising at least one carboxylic acid function,
ethylenically unsaturated monomers comprising one or more silicon atoms, such as methacryloyloxypropyl-trimethoxysilane and methacryloyloxypropyltris-(trimethylsiloxy)silane; and
perfluorooctyl acrylate.

Non-limiting examples of the at least one insoluble monomer includes methyl, ethyl, propyl, n-butyl, cyclohexyl or tert-butyl (meth)acrylate; isobutyl methacrylate; methoxyethyl or ethoxyethyl (meth)acrylate; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate and 2-hydroxyethyl acrylate; dimethylaminopropylmethacrylamide; (meth)acrylic acid; styrene, perfluorooctyl acrylate; and salts thereof.

In a non-limiting embodiment, the at least one insoluble monomer is chosen from methyl acrylates, methoxyethyl acrylates, methyl methacrylates, isobutyl methacrylates, 2-hydroxyethyl methacrylates, acrylic acid, methacrylic acid, dimethylaminoethyl methacrylates, perfluorooctyl acrylates, cyclohexyl methacrylates and tert-butyl acrylates, and salts thereof.

Non-limiting mention is made of copolymers wherein:
the at least one soluble monomer is chosen from isobornyl acrylate, isobornyl methacrylate, isobutyl acrylate and 2-ethylhexyl acrylate, and mixtures thereof; and/or
the insoluble monomer is chosen from isobutyl methacrylate and methyl methacrylate, and mixtures thereof.

In another non-limiting embodiment of the disclosure, the copolymer(s) comprises:
isobornyl acrylate and isobutyl methacrylate;
isobornyl acrylate and methyl methacrylate;
isobornyl methacrylate and isobutyl methacrylate;
isobornyl methacrylate and methyl methacrylate;
isobornyl acrylate, 2-ethylhexyl acrylate; and isobutyl methacrylate;
isobornyl acrylate, isobutyl acrylate and isobutyl methacrylate;
isobornyl methacrylate, isobutyl acrylate and isobutyl methacrylate; or
isobornyl acrylate, 2-ethylhexyl acrylate and methyl methacrylate.

Among the salts, non-limiting mention is made of those obtained by neutralization of acid groups using mineral bases such as LiOH, NaOH, KOH, Ca(OH)₂, NH₄OH or Zn(OH)₂; or with an organic base such as a primary, secondary or tertiary alkylamine, especially triethylamine or butylamine. These primary, secondary or tertiary alkylamines may comprise one or more nitrogen and/or oxygen atoms and therefore may comprise one or more alcohol functions; Non-limiting examples of such alkyamines include 2-amino-2-methylpropanol, triethanolamine, 2-dimethylaminopropanol, lysine, or 3-(dimethylamino)propylamine.

Further non-limiting examples of salts that may be utilized include: salts of mineral acids, such as sulfuric acid, hydrochloric acid, hydrobromic acid, hydroiodic acid, phosphoric acid or boric acid; and salts of organic acids, including those that comprise one or more carboxylic, sulfonic or phosphonic acid groups. These salts may be linear, branched or cyclic aliphatic acids, or aromatic acids. These acids may further comprise one or more hetero atoms chosen from O and N, which may be present, for example, in the form of hydroxyl groups. In a non-limiting embodiment of the invention, the acid is chosen from propionic acid, acetic acid, terephthalic acid, citric acid and tartaric acid.

The soluble monomers as defined above may be present in the copolymer in an amount ranging from 50% to 99% by weight, such as from 60% to 90%, or from 70% to 85% relative to the total weight of monomers.

The insoluble monomers as defined above may be present in the copolymer in an amount ranging from 1% to 50% by weight, such as from 10% to 40%, or from 15% to 30% by weight relative to the total weight of monomers.

The copolymer may, additionally comprise at least one additional monomer, wherein the additional monomer is chosen from one or more insoluble or soluble monomers. In a non-limiting embodiment of the disclosure, the additional monomer is a soluble monomers.

In another non-limiting embodiment of the disclosure, the copolymer comprises at least one monomer with a Tg of less than or equal to 20° C., i.e., from −150° C. to 20° C., from −130° C. to 18° C., or from −120° C. to 15° C., or a mixture of such monomers.

The at least one monomer having the Tg property disclosed above may be chosen from the soluble or insoluble monomers mentioned above.

The at least one monomer with a Tg≦20° C. may be present in the copolymer in an amount ranging from 1% to 99% by weight, such as from 10% to 90%, 20% to 80%, or 25% to 75% by weight, relative to the total weight of the copolymer.

In a non-limiting embodiment, the copolymer according to the disclosure further comprises at least one monomer with a Tg of greater than or equal to 20° C., i.e. from 25 to 150° C., from 30 to 145° C., or from 40 to 140° C., or a mixture of such monomers.

These monomers having a Tg of greater than or equal to 20, 25, 30, and 40° C. may be chosen from the soluble or insoluble monomers mentioned above.

The monomer(s) with a Tg≧20° C. may be present in an amount ranging from 1% to 99% by weight, i.e. from 10 to 90% by weight, from 20 to 80% by weight, or from 25 to 75% by weight, relative to the total weight of the copolymer.

For the purpose of the present disclosure, the term “monomer with a Tg” denotes monomers whose homopolymer has such a Tg. In the present disclosure, the Tg (glass transition temperature) values are theoretical Tg values determined from the theoretical Tg values of the constituent monomers of each of the blocks, which may be found in a reference manual such as the Polymer Handbook, 3^{rd} edition, 1989, John Wiley, according to the following relationship, known as Fox's law:

$$\frac{1}{Tg} = {\sum }_{i} ( \frac{wi}{Tgi} )$$

wherein wi is the mass fraction of the monomer i in the block under consideration and Tgi is the glass transition temperature of the homopolymer of the monomer i.

A person skilled in the art would know how to select the monomers and the amounts thereof as a function of the desired result, on the basis of his general knowledge, for example, of the relative reactivity of each monomer. For example, the type and amount of monomers and the solvent medium may be chosen so as to obtain a copolymer that is soluble in the said solvent medium.

In a non-limiting embodiment of the present disclosure, the copolymers of the present disclosure are soluble in lipophilic solvent media, such as the solvents (i.e., the lipophilic solvents) and/or the carbon-based oils conventionally used in cosmetics.

For the purposes of this disclosure, the term “soluble,” means that the polymer does not form a precipitate in a solvent in which it is contained. In a non-limiting embodiment of the present disclosure, the copolymer is soluble at a concentration of at least 1% by weight in isododecane at 25° C. and 1 atm.

The gradient copolymers of the present disclosure may be present in cosmetic, dermatological, or topical compositions in an amount ranging from 0.1% to 95% by weight, i.e. from 0.5% to 90%, from 1% to 80%, from 5-70%, or from 8% to 30% by weight, relative to the total weight of the composition.

The copolymers of the present disclosure may be present in the composition in dissolved form, for example in an organic solvent or a cosmetic carbon-based oil.

Non-limiting embodiments of the copolymers of the present disclosure may be soluble in cosmetic solvents, and may be used in organic cosmetic media, while at the same time retaining advantageous rheological properties.

The cosmetic or dermatological compositions of the present disclosure may further comprise, in addition to the aforementioned copolymers, a physiologically acceptable medium, such as a cosmetically or dermatologically acceptable medium (i.e., a medium that is compatible with keratin materials such as facial or body skin, the lips, the hair, the eyelashes, the eyebrows and the nails).

The composition of the present disclosure may further comprise a solvent medium, which may be a fatty phase that may itself comprise oils and/or solvents that may be lipophilic, as well as fatty substances that are solid at room temperature, i.e. waxes, pasty fatty substances and gums, and mixtures thereof.

Among the constituents of the fatty phase, non-limiting mention may be made of at least one oil and/or solvent having a global solubility parameter according to the Hansen solubility space of less than or equal to 20 (MPa)^{1/2}, less than or equal to 18 (MPa)^{1/2}, or less than or equal to 17 (MPa)^{1/2}.

The global solubility parameter δ according to the Hansen solubility space is defined in the article “Solubility parameter values” by Eric A. Grulke in the book “Polymer Handbook”, 3^{rd} edition, Chapter VII, pp. 519-559, by the relationship:
δ=(dD²+dP²+dH²)^{1/2}
in which
   dD characterizes the London dispersion forces derived from the formation of dipoles induced during molecular impacts,
   dP characterizes the Debye forces of interaction between permanent dipoles, and
   dH characterizes the specific forces of interaction (such as hydrogen bonding, acid/base, donor/acceptor, etc.).

The definition of solvents in the Hansen solubility space is described in the article by C. M. Hansen “The three-dimensional solubility parameters”, J. Paint Technol. 39, 105 (1967).

Among the at least one oil and/or solvent having a global solubility parameter according to the Hansen solubility space of less than or equal to 20 (MPa)^{1/2}, non-limiting mention may be made of branched or unbranched, volatile or non-volatile oils, which may be chosen from natural or synthetic oils, carbon-based oils, hydrocarbon-based oils, fluoro oils, and mixtures thereof; ethers and esters containing at least 6 carbon atoms, such as from C6 to C30 ethers and esters; ketones containing at least 6 carbon atoms, such as C6 to C30 ketones; C6 to C30 aliphatic fatty monoalcohols, the hydrocarbon-based chain of which does is un substituted.

For the purpose of the present disclosure, the term “non-volatile oil” means an oil that is capable of remaining on the skin at room temperature and atmospheric pressure for at least one hour, and which may have vapor pressure at room temperature (25° C.) and atmospheric pressure, of less than 0.01 mm Hg (1.33 Pa).

Of the aforementioned non-volatile oils, non-limiting mention may be made of carbon-based oils, including hydrocarbon-based non-volatile oils of plant, mineral, animal or synthetic origin, such as liquid paraffin (petroleum jelly), squalane, hydrogenated polyisobutylene (PARLEAM oil), perhydrosqualene, mink oil, macadamia oil, turtle oil, soybean oil, sweet almond oil, beauty-leaf oil, palm oil, grapeseed oil, sesame seed oil, corn oil, arara oil, rapeseed oil, sunflower oil, cottonseed oil, apricot oil, castor oil, avocado oil, jojoba oil, olive oil or cereal germ oil, and shea butter oil; linear, branched or cyclic esters containing more than 6 carbon atoms, for example C6 to C30 esters such as lanolic acid, oleic acid, lauric acid or stearic acid esters; esters derived from long-chain acids or alcohols (i.e. acids or alcohols containing from 6 to 20 carbon atoms), such as esters of formula RCOOR′, in which R represents a C7-C19 higher fatty acid residue and R′ represents a C3-C20 hydrocarbon-based chain, for example C12-C36 esters such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, bis(2-ethylhexyl) succinate, diisostearyl malate and glyceryl or diglyceryl triisostearate; higher fatty acids, including C14-C22 fatty acids such as myristic acid, palmitic acid, stearic acid, behenic acid, oleic acid, linoleic acid, linolenic acid or isostearic acid; higher fatty alcohols, such as C16-C22 higher fatty alcohols, such as cetanol, oleyl alcohol, linoleyl alcohol, linolenyl alcohol, isostearyl alcohol or octyldodecanol; and mixtures thereof.

Non-limiting mention may also be made of decanol, dodecanol, octadecanol, liquid triglycerides of C4-C10 fatty acids, such as heptanoic or octanoic acid triglycerides, and caprylic/capric acid triglycerides; linear or branched hydrocarbons of mineral or synthetic origin, such as liquid paraffins and derivatives thereof, petroleum jelly, polydecenes, and hydrogenated polyisobutene such as PARLEAM; synthetic esters and ethers, including those of fatty acids, such as PURCELLIN oil, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate, and fatty alcohol heptanoates, octanoates and decanoates; polyol esters, such as propylene glycol dioctanoate, neopentyl glycol diheptanoate or diethylene glycol diisononanoate; pentaerythritol esters; and C12 to C26 fatty alcohols, such as octyldodecanol, 2-butyloctanol, 2-hexyldecanol or 2-undecylpentadecanol.

Non-limiting mention may also be made of ketones that are liquid at room temperature, such as methyl ethyl ketone, methyl isobutyl ketone, diisobutyl ketone, isophorone, cyclohexanone or acetone; propylene glycol ethers that are liquid at room temperature, such as propylene glycol monomethyl ether, propylene glycol monomethyl ether acetate or dipropylene glycol mono-n-butyl ether; C3-C8 short-chain esters, such as ethyl acetate, methyl acetate, propyl acetate, n-butyl acetate or isopentyl acetate; ethers that are liquid at room temperature, such as diethyl ether, dimethyl ether or dichlorodiethyl ether; alkanes that are liquid at room temperature, such as decane, heptane, dodecane, isododecane, isohexadecane or cyclohexane; cyclic aromatic compounds that are liquid at room temperature, such as toluene and xylene; aldehydes that are liquid at room temperature, such as benzaldehyde and acetaldehyde; and mixtures thereof.

Among the volatile compounds, non-limiting mention may be made of non-silicone volatile oils, such as C8 to C16 Isoparaffins, including, for example, isododecane, isodecane, and isohexadecane and the oils sold under the trade names ISOPAR and PERMETHYL. In a non-limiting embodiment of the present disclosure, the volatile compound is the isododecane known as PERMETHYL 99A.

In addition, non-limiting mention may be made of volatile or non-volatile alkanes that are liquid at room temperature, such as decane, heptane, dodecane, isododecane, isohexadecane, cyclohexane and isodecane, and mixtures thereof.

These oils and/or solvents may be present in the composition in an amount ranging from 0.01% to 95%, i.e., from 0.1% to 90%, from 10% to 85%, or from 30% to 80% by weight, relative to the total weight of the composition.

The composition of the present disclosure may further comprise a hydrophilic medium comprising water or a mixture of water and at least one hydrophilic organic solvent. Non-limiting examples of suitable compounds that may be used in the hydrophilic medium include alcohols, such as linear or branched lower monoalcohols containing from 2 to 5 carbon atoms, i.e. ethanol, isopropanol or n-propanol; polyols, such as glycerol, diglycerol, propylene glycol, sorbitol, pentylene glycol and polyethylene glycols; hydrophilic C2 ethers; and hydrophilic C2-C4 aldehydes.

The water or the mixture of water and of hydrophilic organic solvents may be present in the composition of the present disclosure in an amount ranging from 0.1% to 80% by weight, or from 1% to 70% by weight, relative to the total weight of the composition.

The composition of the present disclosure may further comprise at least one wax and/or gum.

For the purposes of the present disclosure, the term “wax” means a lipophilic compound that is solid at room temperature (25° C.), can undergo a reversible solid/liquid phase change, and has a melting point ranging from 30° C. to 120° C. By bringing the wax to the liquid state (melting), the wax may become miscible with the oils that may be present in the composition, resulting in the formation of a microscopically homogeneous mixture. However, on returning the temperature of the mixture to room temperature, recrystallization of the wax in the oils of the mixture takes place. The melting point of the wax may be measured using a differential scanning calorimeter (DSC), such as the calorimeter sold under the name DSC 30 by the company Mettler.

Non-limiting examples of compounds which may be utilized as the aforementioned at least one wax include hydrocarbon-based waxes, fluoro waxes and/or silicone waxes. These waxes may be of plant, mineral, animal and/or synthetic origin. The waxes may have a melting point of greater than 25° C., e.g., greater than 45° C. Of these waxes, non-limiting mention may be made of beeswax, carnauba wax, candelilla wax, paraffin, microcrystalline waxes, ceresin or ozokerite; synthetic waxes, such as polyethylene waxes or Fischer Tropsch waxes; and silicone waxes, such as C16 to C45 alkyl or alkoxy dimethicones.

Non-limiting examples of the aforementioned gums include polydimethylsiloxanes (PDMSs) of high molecular weight, cellulose gums, polysaccharides, and pasty substances that are generally hydrocarbon-based compounds, such as lanolins, derivatives thereof, or polydimethylsiloxanes.

The type and amount of the solid substances used in the present disclosure depend on the mechanical properties and textures desired. Non-limiting examples of the composition of the present disclosure may contain from 0.01% to 50% by weight, e.g., from 1% to 30% by weight, of the aforementioned waxes, relative to the total weight of the composition.

The composition of the present disclosure may further comprise one or more dyestuffs chosen from water-soluble dyes, liposoluble dyes and pulverulent dyestuffs, such as pigments, nacres and flakes that are known to those skilled in the art. These dyestuffs may be present in the composition in an amount ranging from 0.01% to 50% by weight, e.g., from 0.01% to 30% by weight, relative to the weight of the composition.

As used herein, the term “pigments” means white or colored, mineral or organic particles of any shape, which are insoluble in the physiological medium and are intended to color the composition. Further, the term “nacres” as used herein, means iridescent particles of any shape, which may be produced synthetically or by certain molluscs in their shell. The pigments may be white or colored, and may be mineral and/or organic. Among these mineral pigments, non-limiting mention may be made of titanium dioxide (which may be surface-treated), zirconium oxide, cerium oxide, zinc oxide, iron oxide (black, yellow or red), chromium oxide, manganese violet, ultramarine blue, chromium hydrate, ferric blue, and metal powders, such as aluminium or copper powder. Among the afroementioned organic pigments, non-limiting mention may be made of carbon black, pigments of D & C type, and lakes based on cochineal carmine or on barium, strontium, calcium or aluminium. Non-limiting examples of nacreous pigments that may be utilized include white nacreous pigments such as mica coated with titanium or bismuth oxychloride, colored nacreous pigments such as titanium mica coated with iron oxides, titanium mica coated with ferric blue or chromium oxide, titanium mica coated with an organic pigment disclosed above, and nacreous pigments based on bismuth oxychloride.

Among water-soluble dyes, non-limiting mention may be made of the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll, and methylene blue.

The composition according to the disclosure may further comprise one or more fillers. Said fillers may be present in the composition in an amount ranging from 0.01% to 50% by weight, e.g., from 0.01% to 30% by weight, relative to the total weight of the composition. As used herein, the term “fillers” means colorless or white, mineral or synthetic particles of any shape, which are insoluble in the medium of the composition irrespective of the temperature at which the composition is manufactured. These fillers may serve to modify the rheology or texture of the composition. The fillers may be mineral or organic and of any shape, i.e. platelet-shaped, spherical or oblong, irrespective of the crystallographic form (for example lamellar, cubic, hexagonal, orthorhombic, etc.). Of these fillers, non-limiting mention may be made of talc, mica, silica, kaolin, polyamide (NYLON) powder (ORGASOL® from Atochem), poly-β-alanine powder, polyethylene powder, powders of tetrafluoroethylene polymers (TEFLON), lauroyllysine, starch, boron nitride, hollow polymer microspheres such as those of polyvinylidene chloride/acrylonitrile, for example EXPANCEL® (Nobel Industrie), acrylic acid copolymers (POLYTRAP from the company Dow Corning), silicone resin microbeads (for example TOSPEARLS® from Toshiba), elastomeric polyorganosiloxane particles, precipitated calcium carbonate, magnesium carbonate, magnesium hydrocarbonate, hydroxyapatite, hollow silica microspheres (SILICA BEADS® from Maprecos), glass or ceramic microcapsules, and metal soaps derived from organic carboxylic acids containing from 8 to 22 carbon atoms, e.g., from 12 to 18 carbon atoms, including, for example zinc, magnesium or lithium stearate, zinc laurate or magnesium myristate.

The composition may further comprise an additional polymer such as a film-forming polymer. According to the present disclosure, the term “film-forming polymer” means a polymer capable of forming, by itself or in the presence of an auxiliary film-forming agent, a continuous film that adheres to a support, i.e. a keratin materials. Among the film-forming polymers that may be used in the composition of the present disclosure, non-limiting mention may be made of synthetic free-radical polymers, polycondensate polymers, and polymers of natural origin and mixtures thereof, i.e. acrylic polymers, polyurethanes, polyesters, polyamides, polyureas and cellulose-based polymers such as nitrocellulose.

The composition according to the disclosure may further comprise additional ingredients that are commonly used in cosmetics, examples of which include, but are not limited to vitamins, thickeners, gelling agents, trace elements, softeners, sequestering agents, fragrances, acidifying or basifying agents, preserving agents, sunscreens, surfactants, antioxidants, agents for preventing hair loss, antidandruff agents, propellants and ceramides, or mixtures thereof. Needless to say, a person skilled in the art will take care to select this or these optional additional compound(s), and/or the amount thereof, such that the advantageous properties of the compositions according to the disclosure are not, or are not substantially, adversely affected by their addition.

The composition according to the disclosure may be in the form of a suspension, a dispersion, a solution (e.g., an organic solution), a gel, an emulsion (e.g., an oil-in-water (O/W); a water-in-oil (W/O) emulsion, or a multiple emulsion (W/O/W or polyol/O/W or O/W/O emulsion)), a cream, a paste, a mousse, a dispersion of vesicles (i.e. of ionic or nonionic lipid vesicles), a two-phase or multi-phase lotion, a spray, a powder or a paste (e.g., a soft paste, such as a paste having a dynamic viscosity at 25° C. of about from 0.1 to 40 Pa·s at a shear rate of 200 s⁻¹, after measurement for 10 minutes in cone/plate geometry). Further, the composition may be anhydrous in form, e.g., as an anhydrous paste.

A person skilled in the art may select the appropriate form of the composition, as well as the method for preparing it, on the basis of his general knowledge, taking into account the nature of the constituents used, such as their solubility in the support, and the intended use of the composition.

The composition according to the disclosure may be a makeup composition, i.e. a complexion product such as a foundation, a makeup rouge, or an eye shadow; a lip product, such as a lipstick or a lip care product; a concealer product; a blush, a mascara or an eyeliner; an eyebrow makeup product such as a lip or eye pencil; a nail product such as a nail varnish or a nail care product; a body makeup product; or a hair makeup product such as a hair mascara or hair lacquer.

The composition of the present disclosure may be a protective or care composition for facial skin, the neck, the hands or the body, such as an anti-wrinkle composition, an anti-fatigue composition for making the skin look radiant, a moisturizing or medicated composition, an sunscreen composition, or an artificial tanning composition.

The composition of the present disclosure may be a hair care product, i.e. for holding a hairstyle or for shaping the hair. The hair care product may be a shampoo, a hair setting gel or lotion, a blow-drying lotion, or a fixing and styling composition such as a lacquer or spray. The lotions may be packaged in various forms, e.g., in vaporizers, pump-dispenser bottles, or aerosol containers to enable application of the composition in vaporized form or in the form of a mousse. Such packaging forms are advantageous when it is desired to obtain a spray or a mousse for fixing or treating the hair.

In a non-limiting embodiment the composition of the present disclosure is a makeup composition, such as a foundation or a lipstick.

Another aspect of the present disclosure is a cosmetic process for making up or caring for keratin materials, such as body or facial skin, the nails, the hair and/or the eyelashes, comprising providing the aforementioned composition, and applying the composition to a keratin material.

In a non-limiting embodiment, the present disclosure relates to a cosmetic process, comprising providing the aforementioned composition in the form of a cosmetic foundation or lipstick, and applying the composition to facial skin or the lips.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The following examples are intended to illustrate the invention in a non-limiting manner.

### EXAMPLE 1

### Preparation of a Gradient Copolymer of poly(isobornyl acrylate)/methyl acrylate Composition

10 g of isododecane, 21 g of isobornyl acrylate, 9 g of methyl acrylate, 1.338 g of iodoperfluorohexane (3×10⁻³ mol) and 0.308 g of PERKADOX SE-10 (9×10⁻⁴ mol) were introduced into a 250 ml three-necked flask. The mixture was stirred at 25° C. and, after dissolution, the three-necked flask was immersed in an oil bath and thermostatically maintained at 80° C. for 1 hour and 20 minutes, after which the degree of conversion observed was 83%.

A gradient copolymer of poly(isobornyl acrylate/methyl acrylate) composition was obtained, with an iodine atom at the end of each chain. The number-average molar mass (Mn) was 8800 g/mol (with a theoretical Mn of 6500 g/mol).

The iodo end functions were characterized by MALDI-TOF or by NMR.

### EXAMPLE 2

### Preparation of a Gradient Copolymer of poly(isobornyl acrylate/methyl acrylate) Composition

10 g of isododecane, 24 g of isobornyl acrylate, 6 g of methyl acrylate, 1.339 g of iodoperfluorohexane (3×10⁻³ mol) and 0.308 g of PERKADOX SE-10 (9×10⁻⁴ mol) were introduced into a 250 ml three-necked flask. The mixture was stirred at 25° C. and, after dissolution, the three-necked flask was immersed in an oil bath and thermostatically maintained at 80° C. for 1 hour and 40 minutes, after which the degree of conversion observed was 81%.

A linear and gradient copolymer of poly(isobornyl acrylate/methyl acrylate) composition was obtained, with an iodine atom at the end of each chain.

The number-average molar mass (Mn) was 7650 g/mol (with a theoretical Mn of 6300 g/mol).

The iodo end functions were characterized by MALDI-TOF or by NMR.

### EXAMPLE 3

### Preparation of a Gradient Copolymer of poly(isobornyl acrylate/methyl acrylate) Composition

10 g of isododecane, 15 g of isobornyl acrylate, 15 g of methyl acrylate, 1.339 g of iodoperfluorohexane (3×10⁻³ mol) and 0.308 g of PERKADOX SE-10 (9×10⁻⁴ mol) were introduced into a 250 ml three-necked flask. The mixture was stirred at 25° C. and, after dissolution, the three-necked flask was immersed in an oil bath and thermostatically maintained at 75° C. for 1 hour 40 minutes, after which the degree of conversion observed was 79%.

A gradient copolymer of poly(isobornyl acrylate/methyl acrylate) composition was obtained, with an iodine atom at each chain end. The number-average molar mass (Mn) obtained was 9500 g/mol (with a theoretical Mn of 6100 g/mol).

The iodo end functions were characterized by MALDI-TOF or by NMR.

### EXAMPLE 4

An anhydrous foundation was prepared, comprising (% by weight):

| | |
|---|---|
| polyethylene wax | 12% |
| volatile silicone oils | 25% |
| phenyltrimethicone | 20% |
| polymethyl methacrylate microspheres | 12% |
| polymer of Example 1 | 6% |
| isododecane | qs |
| | 100% |

Preparation:

The waxes were melted and, when clear, the phenyl trimethicone was added with stirring, along with the silicone oils; the microspheres, the isododecane and the polymer were then added. The mixture was homogenized for 15 minutes and the resulting composition was cast and allowed to cool.

An anhydrous foundation was obtained.

### EXAMPLE 5

A lipstick was prepared, comprising:

| | |
|---|---|
| polyethylene wax | 15% |
| polymer of Example 2 | 10% |
| | AM |
| hydrogenated polyisobutene (PARLEAM | 25% |
| from Nippon Oil Fats) | |
| pigments | 10% |
| isododecane | qs |
| | 100% |

The composition that was obtained exhibited good cosmetic properties after application to the lips.

### EXAMPLE 6

### W/O Foundation

A foundation composition comprising the following compounds was prepared:

Phase A

| Phase A | | |
|---|---|---|
| cetyl dimethicone copolyol (Abil EM 90 | 3 | g |
| from Goldschmidt) | | |
| isostearyl diglyceryl succinate (IMWITOR | 0.6 | g |
| 780 K from the company Condea) | | |
| isododecane | 18.5 | g |
| pigments (iron oxide and titanium oxide) | 10 | g |
| polymer of Example 1 | 8.7 | g |
| | ADM | |
| polyamide powder (NYLON-12) | 8 | g |

| fragrance | qs | |
|---|---|---|
| Phase B | | |
| water | qs | |
| | 100 | g |
| magnesium sulphate | 0.7 | g |

| | |
|---|---|
| preserving agent | qs |

ADM: active dry material

The composition that was obtained exhibited good cosmetic properties.

### EXAMPLE 7

### Preparation of a Block Copolymer of Polystyrene-b-Poly(styrene/isobornyl acrylate) Composition

6 g of styrene, 0.0337 g of PERKADOX SE-10 and 0.1467 g of iodoperfluorohexane were introduced into a 10 ml tube. The tube was immersed in a bath at 80° C. for 3 hours, after which the styrene conversion was 95% and the number-average molar mass (Mn) observed was 8345 g/mol (lp=2.16). 3 g of isobornyl acrylate and 0.0168 g of PERKADOX SE-10 were then introduced into the tube. The tube was then immersed in an oil bath at 90° C. for 4 hours.

A gradient copolymer of polystyrene-b-poly(styrene/isobornyl acrylate) composition was obtained, with an iodine atom at the end of each chain.

The copolymer that was obtained had a number-average molar mass (Mn) of 16,700 g/mol (Ip=1.90).

Shift of the UV peak of the final copolymer relative to the peak of the first block confirmed the formation of a block copolymer.

The iodo end functions were characterized by MALDI-TOF or by NMR.

What is claimed is:

1. A copolymer having at least two ends, wherein at least one end is functionalized with an iodine atom, said copolymer comprising at least two different monomers, wherein one of the at least two different monomers is chosen from at least one soluble monomer having a homopolymer solubility of at least 3% by weight in isododecane at 25° C. and 1 atm, and the other of the at least two different monomers is chosen from at least one insoluble monomer having a homopolymer solubility of less than 3% by weight in isododecane at 25° C. and 1 atm.

2. The copolymer of claim 1, wherein the copolymer is obtained by free-radical polymerization controlled by degenerative transfer at the iodine atom from at least one of:
at least one free-radical polymerization initiator;
at least one transfer agent comprising at least one iodine atom; and
at least two different monomers.

3. The copolymer of claim 1, wherein the at least one soluble monomer is chosen from:
(i) (meth)acrylates of formula CH₂═CHCOOR or CH₂═C(CH₃)COOR, wherein R represents:
a linear or branched C8 to C30 alkyl group, wherein said alkyl group is optionally: a) intercalated with one or more hetero atoms chosen from O, N, S, and P; or b) substituted with one or more substituents chosen from —OH, halogen atoms, or —NR₄R₅ groups, in which R₄ and R₅ may be identical or different and represent hydrogen, a linear or branched C1 to C6 alkyl group, or a phenyl group;
a C8 to C12 cycloalkyl group,
(ii) tert-butyl methacrylate and isobutyl acrylate;
(iii) (meth)acrylamides of formula CH₂═CHCONR₄R₅ or CH₂═C(CH₃)CONR₄R₅ in which:
R₄ represents a hydrogen atom or a linear or branched C1 to C6 alkyl group;
R₅ represents a linear or branched C8 to C18 alkyl group;
(iv) vinyl ethers of formula R₆O—CH═CH₂ or vinyl esters of formula: R₆—COO—CH═CH₂ in which R₆ represents a linear or branched C8 to C22 alkyl group;
(v) carbon-based macromonomers having at least one polymerizable end group, obtained from soluble monomers having a homopolymer solubility of at least 3% by weight in isododecane at 25° C. and 1 atm;
(vi) C2 to C10 ethylenic hydrocarbons;
and salts thereof.

4. The copolymer of claim 3, wherein said halogens are chose from Cl, I, Br, and F.

5. The copolymer of claim 3, wherein R is chosen from octyl, decyl, lauryl, isooctyl, isodecyl, dodecyl, t-butylcyclohexyl, stearyl, 2-ethylhexyl, isobornyl and behenyl groups.

6. The copolymer of claim 3, wherein R₄ is chosen from: hydrogen, methyl, ethyl, propyl, isopropyl, n-butyl, isobutyl, tert-butyl, hexyl, isohexyl, and cyclohexyl groups.

7. The copolymer of claim 3, wherein R₅ is chosen from: octyl, isooctyl, decyl, isodecyl, cyclodecyl, dodecyl, cyclododecyl, isononyl, undecyl, lauryl, t-butylcyclohexyl, stearyl; and 2-ethylhexyl groups.

8. The copolymer of claim 3, wherein said ethylenic hydrocarbons are chosen from ethylene, isoprene and butadiene.

9. The copolymer of claim 3, wherein said carbon-based macromonomers are chosen from:
a) linear or branched C8-C22 alkyl (meth)acrylate homopolymers and copolymers containing at least one polymerizable end group chosen from vinyl or (meth)acrylate groups; and
b) polyolefins containing an ethylenically unsaturated end group.

10. The copolymer of claim 9, wherein said linear or branched C8-C22 alkyl (meth)acrylate homopolymers and copolymers are chosen from: poly(2-ethylhexyl acrylate) macromonomers containing a mono(meth)acrylate end; poly(dodecyl acrylate) or poly(dodecyl methacrylate) macromonomers containing a mono(meth)acrylate end; poly(stearyl acrylate); and poly(stearyl methacrylate) macromonomers containing a mono(meth)acrylate end.

11. The copolymer of claim 9, wherein said polyolefins containing an ethylenically unsaturated end group comprise a methacrylate end group.

12. The copolymer of claim 11, wherein said polyolefins containing an ethylenically unsaturated end group are chosen from: polyethylene macromonomers; polypropylene macromonomers; polyethylene/polypropylene copolymer macromonomers; polyethylene/polybutylene copolymer macromonomers; polyisobutylene macromonomers; polybutadiene macromonomers; polyisoprene macromonomers; polybutadiene macromonomers; and poly(ethylene/butylene)-polyisoprene macromonomers.

13. The copolymer of claim 1, wherein the soluble monomer is chosen from:
a) (meth)acrylates having the formula CH₂═CHCOOR or CH₂═C(CH₃)COOR, in which R represents a linear or branched C8 to C30 alkyl group; a C8 to C12 cycloalkyl group;
b) linear or branched C8 to C22 alkyl (meth)acrylate homopolymers and copolymers containing a polymerizable end group chosen from vinyl or (meth)acrylate groups containing at least one polymerizable end group; and
c) polyolefins containing an ethylenically unsaturated end group.

14. The copolymer of claim 13, wherein said polyolefins containing an ethylenically unsaturated end group comprise a (meth)acrylate end group.

15. The copolymer of claim 1, wherein said soluble monomer is chosen from:
isobornyl acrylate, isobornyl methacrylate, 2-ethylhexyl acrylate, 2-ethylhexyl methacrylate, lauryl acrylate, lauryl methacrylate, stearyl acrylate, stearyl methacrylate, tert-butyl methacrylate, isobutyl acrylate;
behenyl methacrylate, behenyl acrylate;
poly(2-ethylhexyl acrylate) macromonomers containing a mono(meth)acrylate end group;
poly(dodecyl acrylate) or poly(dodecyl methacrylate) macromonomers containing a mono(meth)acrylate end group;
polystearyl (meth)acrylate macromonomers containing a mono(meth)acrylate end group; and
ethylene/butylene or ethylene/propylene macromonomers containing a (meth)acrylate reactive end group.

16. The copolymer of claim 1, wherein said insoluble monomer is chosen from:
(i) (meth)acrylates of the formula CH₂═CHCOOR or CH₂═C(CH₃)COOR, in which R represents:
a linear or branched C1 to C6 alkyl group, with the exception of tert-butyl methacrylate and isobutyl acrylate, wherein said alkyl group is optionally: a) intercalated with one or more hetero atoms chosen from O, N, S and P; or b) substituted with one or more substituents chosen from —OH, halogen atoms, or —NR₄R₅ groups, in which R₄ and R₅ may be identical or different, and represent hydrogen, a linear or branched C1 to C6 alkyl group, a phenyl group, or a polyoxyalkylene group comprising from 5 to 30 repeating oxyalkylene units;
a C3 to C6 cycloalkyl group that: a) optionally comprises one or more hetero atoms chosen from O, N, S and/or P in its chain; or b) may be substituted with one or more substituents chosen from —OH and halogen atoms;
a C5 to C8 aryl or a C6 to C10 aralkyl group having a C1 to C5 alkyl group;
(ii) (meth)acrylamides of the formula CH₂═CHCONR₄R₅ or CH₂═C(CH₃)CONR₄R₅
in which R₄ and R₅ may be identical or different, and represent a hydrogen atom or a linear or branched C1 to C6 alkyl group that is optionally: a) intercalated one or more hetero atoms chosen from O, N, S and P; or b) substituted with one or more substituents chosen from hydroxyl groups, halogen atoms and —NR₁₄R₁₅ groups, in which R′₄ and R′₅ may be identical or different, and represent a C1 to C4 alkyl group;
(iii) ethylenically unsaturated monomers comprising at least one carboxylic, phosphoric or sulfonic acid or anhydride function
(iv) vinyl ethers of the formula R₆O—CH═CH₂ or the vinyl esters of formula R₆—COO—H═CH₂ in which R₆ represents a C1 to C6 linear or branched alkyl group;
(v) styrene and its derivatives;
(vi) silicone macromonomers;
(vii) ethylenically unsaturated monomers comprising at least one tertiary amine function;
(viii) vinyl compounds of formula: CH2═CH—R₉, CH2═CH—CH2-R₉ or CH2═C(CH3)-CH2-R₉
in which R₉ is a hydroxyl group, a halogen atom, an NH₂ group, or an —OR₁₀ group, in which R₁₀ is a phenyl group or a C1 to C12 alkyl group;
(ix) ethylenically unsaturated monomers comprising one or more silicon atoms;
(x) perfluorooctyl acrylate.

17. The copolymer of claim 16, wherein R is chosen from methyl, ethyl, propyl, isopropyl, n-butyl, isobutyl, tert-butyl, hexyl, cyclohexyl, 2-hydroxyethyl, 2-hydroxybutyl, 2-hydroxypropyl, methoxyethyl, ethoxyethyl, methoxypropyl, phenyl, 2-phenylethyl, t-butylbenzyl, benzyl, furfurylmethyl or tetrahydrofurfurylmethyl, methoxypolyoxyethylene (or POE-methyl) groups; POE-behenyl, trifluoroethyl; dimethylaminoethyl, diethylaminoethyl, and dimethylaminopropyl groups.

18. The copolymer of claim 16, wherein R₄ represents a hydrogen atom and R₅ represents a 1,1-dimethyl-3-oxobutyl group.

19. The copolymer of claim 16, wherein R₄ and R₅ are chosen from: hydrogen, methyl, ethyl, propyl, isopropyl, n-butyl, isobutyl, tert-butyl, hexyl, isohexyl, cyclohexyl, dimethylaminoethyl, diethylaminoethyl, and dimethylaminopropyl groups.

20. The copolymer of claim 16, wherein said ethylenically unsaturated monomers comprising at least one carboxylic, phosphoric or sulfonic acid or anhydride function are chosen from acrylic acid, methacrylic acid, crotonic acid, maleic anhydride, itaconic acid, fumaric acid, maleic acid, styrenesulfonic acid, vinylbenzoic acid, vinylphosphoric acid, acrylamidopropanesulfonic acid, and the salts thereof.

21. The copolymer of claim 16, wherein the styrene and styrene derivatives are chosen from methylstyrene, chlorostyrene and chloromethylstyrene.

22. The copolymer of claim 16, wherein said silicone macromonomer is a polydimethylsiloxane containing a mono(meth)acrylate end group.

23. The copolymer of claim 22, wherein said silicone macromonomer is of the formula (IIa) below: 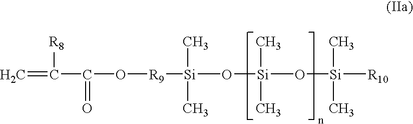
in which:
R₈ denotes a hydrogen atom or a methyl group;
R₉ denotes a linear or branched C1 to C10 divalent hydrocarbon-based group that may comprise one or two ether bonds;
R₁₀ denotes a linear or branched C1 to C10 alkyl group;
and n denotes an integer ranging from 1 to 300.

24. The copolymer of claim 16, wherein said vinyl compound is chosen from 2-vinylpyridine or 4-vinylpyridine.

25. The copolymer of claim 16, wherein said ethylenically unsaturated monomers comprising one or more silicon atoms are chosen from methacryloyloxypropyltrimethoxysilane and methacryloyloxypropyltris(trimethylsiloxy)silane.

26. The copolymer of claim 1, wherein the insoluble monomer is chosen from:
(meth)acrylates of formula CH₂═CHCOOR or CH₂═C(CH₃)COOR in which R represents, with the exception of tert-butyl methacrylate and isobutyl acrylate:
a linear or branched C1 to C6 alkyl or C3 to C6 cycloalkyl group;
a (meth)acrylamide of the formula CH₂═CHCONR₄R₅ or CH₂═C(CH₃)CONR₄R₅
in which R₄ and R₅ may be identical or different, and represent a hydrogen atom or a linear or branched C1 to C6 alkyl group;
an ethylenically unsaturated monomer comprising at least one carboxylic acid function;
an ethylenically unsaturated monomer comprising one or more silicon atoms; or
a perfluorooctyl acrylate.

27. The copolymer of claim 26, wherein R₄ represents a hydrogen atom and R₅ represents a 1,1-dimethyl-3-oxobutyl group.

28. The copolymer of claim 26, wherein said ethylenically unsaturated monomers comprising one or more silicon monomers are chosen from methacryloyloxypropyl-trimethoxysilane and methacryloyloxypropyltris(trimethylsiloxy)silane.

29. The copolymer of claim 1, wherein the at least one insoluble monomer is chosen from methyl, ethyl, propyl, n-butyl, cyclohexyl or tert-butyl (meth)acrylate; isobutyl methacrylate; methoxyethyl or ethoxyethyl (meth)acrylate; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate and 2-hydroxyethyl acrylate; dimethylaminopropylmethacrylamide; (meth)acrylic acid; styrene, perfluorooctyl acrylate; and salts thereof.

30. The copolymer of claim 1, wherein the at least one insoluble monomer is chosen from methyl acrylate, methoxyethyl acrylate, methyl methacrylate, isobutyl methacrylate, 2-hydroxyethyl methacrylate, acrylic acid, methacrylic acid, dimethylaminoethyl methacrylate, perfluorooctyl acrylate, cyclohexyl methacrylate, tert-butyl acrylate, and salts thereof.

31. The copolymer of claim 1, wherein:
the at least one soluble monomer is chosen from isobornyl acrylate, isobornyl methacrylate, isobutyl acrylate, and 2-ethylhexyl acrylate; or
the at least one insoluble monomer is chosen from isobutyl methacrylate and methyl methacrylate.

32. The copolymer of claim 1, wherein:
the at least one soluble monomer is chosen from isobornyl acrylate, isobornyl methacrylate, isobutyl acrylate, and 2-ethylhexyl acrylate; and
the at least one insoluble monomer is chosen from isobutyl methacrylate and methyl methacrylate.

33. The copolymer of claim 1, wherein said at least one soluble monomer is present in an amount ranging from 50% to 99% by weight, relative to the total weight of monomers in the copolymer.

34. The copolymer of claim 33, wherein said at least one soluble monomer is present in an amount ranging from 70% to 85% by weight, relative to the total weight of monomers in the composition.

35. The copolymer of claim 1, wherein said at least one insoluble monomer is present in an amount ranging from 1% to 50% by weight, relative to the total weight of monomers in the composition.

36. The copolymer of claim 35, wherein said at least one insoluble monomer is present in an amount ranging from 15% to 30% by weight, relative to the total weight of monomers in the composition.

37. The copolymer of claim 1, comprising at least one monomer having a Tg of less than or equal to 20° C. relative to the total weight of the copolymer.

38. The copolymer of claim 37, comprising at least one monomer having a Tg from −120° C. to 15° C.

39. The copolymer of claim 37, wherein said at least one monomer having a Tg of less than or equal to 20° C. is present in an amount ranging from 1% to 99% by weight relative to the total weight of the copolymer.

40. The copolymer of claim 39, wherein said at least one monomer having a Tg of less than or equal to 20° C. is present in an amount ranging from 25% to 75% by weight relative to the total weight of the copolymer.

41. The copolymer of claim 1, wherein said copolymer comprises at least one monomer with a Tg of greater than or equal to 20° C.

42. The copolymer of claim 41, wherein said copolymer comprises at least one monomer having a Tg from 30 and 145° C.

43. The copolymer of claim 41, wherein said at least one monomer having a Tg greater than or equal to 20° C. is present in an amount ranging from 1% to 99% by weight, relative to the total weight of the copolymer.

44. The copolymer of claim 43, wherein said at least one monomer having a Tg greater than or equal to 20° C. is present in an amount ranging from 25% to 75% by weight, relative to the total weight of the copolymer.

45. The copolymer of claim 1, wherein said copolymer is soluble at a concentration of at least 3% by weight in isododecane at 25° C. and 1 atm.

46. The copolymer of claim 45, wherein said copolymer is soluble at a concentration of at least 10% by weight in isododecane at 25° C. and 1 atm.

47. The copolymer of claim 1, wherein said copolymer is a block polymer or a gradient polymer.

48. The copolymer of claim 1, wherein said copolymer has a linear-chain, grafted, or star structure.

49. The copolymer of claim 1, wherein the copolymer has a number-average molecular mass ranging from 2000 g/mol to 1,000,000 g/mol.

50. The copolymer of claim 49, wherein the copolymer has a number-average molecular mass ranging from 5000 g/mol to 50,000 g/mol.

51. The copolymer of claim 2, wherein the at least one free radical polymerization initiator is chosen from: azo compounds; peroxide compounds; redox couples, peresters, percarbonates and persulfates; 2,5-bis(2-ethylhexanoylperoxy)-2,5-dimethylhexane; and tert-butyl peroxy-2-ethylhexanoate.

52. The copolymer of claim 51, wherein said azo compounds are azobisisobutyronitriles.

53. The copolymer of claim 51, wherein said peroxide compounds are chosen from organic hydroperoxides or C6-C30 peroxide compounds.

54. The copolymer of claim 53, wherein said peroxide compounds are chosen from benzoyl or didecanoyl peroxides.

55. The copolymer of claim 2, wherein the at least one transfer agent is represented by the formula R-I, in which R is a linear, branched or cyclic, saturated or unsaturated C1 to C30 alkyl radical that may further comprise 1 to 4 additional iodine atoms, one or more fluorine atoms, and/or one or more functional groups chosen from —CN and —COOH.

56. The copolymer according of claim 55, wherein R is chosen from C1 to C6 alkyls that may further comprise one or more halogen atoms and/or a —CN functional group.

57. The copolymer according to claim 55, wherein the at least one transfer agent is chosen from iodo-1-perfluorohexane, iodoacetonitrile (ICH₂—CN), iodo-1-methane, diiodomethane, iodoform or triiodomethane, iodo-1-perfluoroisopropane, diiodoperfluorohexane, iodo-1-phenylethane, iodo-1-propane, iodo-1-isopropane, iodo-1-phenyl, 1,4-diiodophenyl and iodo-1-tert-butane.

58. The copolymer of claim 2, wherein the at least one transfer agent is macromolecular, is in the form of a polymer, homopolymer or copolymer, and is functionalized with at least one iodine atom.

59. A cosmetic or dermatological composition comprising, in a physiologically acceptable medium, at least one copolymer having at least two ends, wherein at least one end is functionalized with an iodine atom, said copolymer comprising at least two different monomers, wherein one of the at least two different monomers is chosen from at least one soluble monomers having a homopolymer solubility of at least 3% by weight in isododecane at 25° C. and 1 atm, and the other of the at least two different monomers is chosen from at least one insoluble monomers having a homopolymer solubility of less than 3% by weight in isododecane at 25° C. and 1 atm.

60. The composition of claim 59, wherein the copolymer is present in the composition in an amount ranging from 0.1% to 95% by weight relative to the total weight of the composition.

61. The composition of claim 60, wherein the copolymer is present in the composition in an amount ranging from 8% to 30% by weight relative to the total weight of the composition.

62. The composition of claim 59, further comprising a fatty phase, said fatty phase optionally comprising oils and/or solvents having a global solubility parameter according to the Hansen solubility space of less than or equal to 20 (MPa)^{1/2}.

63. The composition of claim 62, wherein said fatty phase has a global solubility parameter according to the Hansen solubility space of less than or equal to 17 (MPa)^{1/2}.

64. The composition of claim 62, further comprising at least one constituent chosen from carbon-based oils, hydrocarbon-based oils fluoro oils, or mixtures thereof, wherein said oils are volatile or non-volatile, natural or synthetic, and optionally branched; ethers and esters containing more than 6 carbon atoms; ketones containing more than 6 carbon atoms; and aliphatic fatty monoalcohols containing 6 to 30 carbon atoms, wherein the hydrocarbon-base of said aliphatic fatty monoalcohols is unsubstituted.

65. The composition of claim 64, wherein said ethers and esters contain from 6 to 30 carbon atoms.

66. The composition of claim 64, wherein said ketones contain from 6 to 30 carbon atoms.

67. The composition of claim 64, comprising at least one oil chosen from carbon-based oils; linear, branched or cyclic esters containing more than 6 carbon atoms; higher fatty acids; higher fatty alcohols; decanol, dodecanol, octadecanol, and liquid triglycerides of C4 to C10 fatty acids; linear or branched hydrocarbons of mineral or synthetic origin;
synthetic esters and ethers; hydroxylated esters; polyol esters; pentaerythritol esters; C12 to C26 fatty alcohols; ketones that are liquid at room temperature; propylene glycol ethers that are liquid at room temperature; C3 to C8 esters; ethers that are liquid at room temperature; alkanes that are liquid at room temperature; cyclic aromatic compounds that are liquid at room temperature; aldehydes that are liquid at room temperature; and non-silicone volatile oils.

68. The composition of claim 67, wherein said carbon based oil is a hydrocarbon based oil chosen from non-volatile oils of plant, mineral, animal or synthetic origin.

69. The composition of claim 68 wherein said hydrocarbon based oil is chosen from one or more of liquid paraffin, squalane, hydrogenated polyisobutylene, perhydrosqualene, mink oil, macadamia oil, turtle oil, soybean oil, sweet almond oil, beauty-leaf oil, palm oil, grapeseed oil, sesame seed oil, corn oil, arara oil, rapeseed oil, sunflower oil, cottonseed oil, apricot oil, castor oil, avocado oil, jojoba oil, olive oil or cereal germ oil, and shea buffer oil.

70. The composition of claim 67, wherein said linear, branched or cyclic esters contain from 6 to 30 carbon atoms.

71. The composition of claim 70, wherein said linear, branched, or cyclic esters are chosen from lanolic acidoleic acid, lauric acid esters; stearic acid esters; and esters derived from C6 to C20 acids or alcohols.

72. The composition of claim 71, wherein said linear, branched, or cyclic esters are of the formula RCOOR′, in which R represents a higher fatty acid residue containing from 7 to 19 carbon atoms and R′ represents a hydrocarbon-based chain containing from 3 to 20 carbon atoms.

73. The composition of claim 67, wherein the linear, branched, or cyclic esters are chosen from C12-C36 esters.

74. The composition of claim 73, wherein the linear, branched, or cyclic esters are chosen from isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, bis(2-ethylhexyl) succinate, diisostearyl malate and glyceryl and diglyceryl triisostearate.

75. The composition of claim 67, wherein said fatty acids contain from 14 to 22 carbon atoms.

76. The composition of claim 75, wherein said fatty acids are chosen from myristic acid, palmitic acid, stearic acid, behenic acid, oleic acid, linoleic acid, linolenic acid and isostearic acid.

77. The composition of claim 67, wherein said higher fatty alcohols contain from 16 to 22 carbon atoms.

78. The composition of claim 77, wherein said higher fatty alcohols are chosen from cetanol, oleyl alcohol, linoleyl alcohol, linolenyl alcohol, isostearyl alcohol, octyldodecanol, and mixtures thereof.

79. The composition of claim 67, wherein said C4 to C10 fatty acids are chosen from heptanoic, octanoic, or caprylic/capric acid triglycerides.

80. The composition of claim 67, wherein said linear or branched hydrocarbons of mineral or synthetic origin are chosen from petroleum jelly, polydecenes, and hydrogenated polyisobutene.

81. The composition of claim 67, wherein said synthetic esters and ethers are chosen from PURCELLIN oil, isopropyl myristate, 2-ethylhexyl palmitate, 2-octyldodecyl stearate, 2-octyldodecyl erucate, and isostearyl isostearate.

82. The composition of claim 67, wherein said hydroxylated esters are chosen from isostearyl lactate, octyl hydroxystearate, octyldodecyl hydroxystearate, diisostearyl malate, triisocetyl citrate, and fatty alcohol heptanoates, octanoates, and decanoates.

83. The composition of claim 67, wherein said polyol esters are chosen from propylene glycol dioctanoate, neopentyl glycol diheptanoate, and diethylene glycol diisononanoate.

84. The composition of claim 67, wherein said C12 to C26 fatty alcohols are chosen from octyldodecanol, 2-butyloctanol, 2-hexyldecanol or 2-undecylpentadecanol.

85. The composition of claim 67, wherein said ketones that are liquid at room temperature are chosen from methyl ethyl ketone, methyl isobutyl ketone, diisobutyl ketone, isophorone, cyclohexanone, and acetone.

86. The composition of claim 67, wherein said propylene glycol ethers that are liquid at room temperature are chosen from propylene glycol monomethyl ether, propylene glycol monomethyl ether acetate, and dipropylene glycol mono-n-butyl ether.

87. The composition of claim 67, wherein said C3 to C8 esters are chosen from ethyl acetate, methyl acetate, propyl acetate, n-butyl acetate, and isopentyl acetate.

88. The composition of claim 67, wherein said ethers that are liquid at room temperature are chosen from diethyl ether, dimethyl ether, and dichlorodiethyl ether.

89. The composition of claim 67, wherein said alkanes that are liquid at room temperature are chosen from decane, heptane, dodecane, isododecane, isohexadecane, and cyclohexane.

90. The composition of claim 67, wherein said cyclic aromatic compounds that are liquid at room temperature are chosen from toluene and xylene.

91. The composition of claim 67, wherein said aldehydes that are liquid at room temperature are chosen from benzaldehyde, acetaldehyde, and mixtures thereof.

92. The composition of claim 67, wherein said nonsilicone volatile oils are chosen from C8-C16 isoparaffins.

93. The composition of claim 92, wherein said nonsilicone volatile oils are chosen from isododecane, isodecane or isohexadecane.

94. The composition of claim 64, comprising at least one constituent chosen from volatile or non-volatile alkanes that are liquid at room temperature.

95. The composition of claim 94, wherein said volatile or non-volatile alkanes are chosen from decane, heptane, dodecane, isododecane, isohexadecane, cyclohexane and isodecane, and mixtures thereof.

96. The composition of claim 62, wherein the oils and/or solvents are present in an amount ranging from 0.01% to 95% by weight, relative to the total weight of the composition.

97. The composition of claim 96, wherein the oils and/or solvents are present in an amount ranging from 30 to 80% by weight, relative to the total weight of the composition.

98. The composition of claim 59, further comprising at least one constituent chosen from water; alcohols; hydrophilic C2 ethers and C2-C4 aldehydes; waxes and/or gums; dyestuffs chosen from water-soluble dyes, liposoluble dyes and pulverulent dyestuffs, fillers, and additional polymers; vitamins, thickeners, gelling agents, trace elements, softeners, sequestering agents, fragrances, acidifying or basifying agents, preserving agents, sunscreens, surfactants, antioxidants, agents for preventing hair loss, antidandruff agents, propellants and ceramides; and mixtures thereof.

99. The composition of claim 98, wherein said alcohols are chosen from linear or branched lower C2 to C5 monoalcohols or polyols.

100. The composition of claim 99, wherein said alcohols are chosen from ethanol, isopropanol, n-propanol, glycerol, diglycerol, propylene glycol, sorbitol, pentylene glycol, and polyethylene glycols.

101. The composition of claim 98, wherein said dyestuffs are chosen from pigments, nacres and flakes.

102. The composition of claim 98, wherein said additional polymers are chosen from film-forming polymers.

103. The composition of claim 59, wherein said composition is in the form of a suspension, a dispersion, a solution, a gel, an emulsion, a cream, a paste, a mousse, a dispersion of vesicles, a multi-phase lotion, a spray, a powder, or a paste; said composition being in anhydrous form.

104. The composition of claim 103, wherein said solution is an organic solution.

105. The composition of claim 103, wherein said emulsion is an oil-in-water emulsion, a water-in-oil emulsion, or a multiple emulsion.

106. The composition of claim 59, wherein the composition is in the form of a makeup composition; a lip product; a concealer product; a blush; a mascara; an eyeliner; an eyebrow makeup product; a lip or eye pencil; a nail product; a body makeup product; a hair makeup product; a protective or care composition for facial skin, the neck, the hands or the body; an anti-fatigue composition for making the skin look radiant; a moisturizing or medicated composition; a sunscreen composition; an artificial tanning composition; or a hair care composition.

107. The composition of claim 106, wherein said makeup composition is a foundation, a makeup rouge, or an eye shadow.

108. The composition of claim 106, wherein said lip product is a lipstick or a lip care product.

109. The composition of claim 106, wherein said nail composition is a nail varnish or a nail care product.

110. The composition of claim 106, wherein said hair makeup product is a hair mascara or hair lacquer.

111. The composition of claim 106, wherein said protective or care composition is an anti-wrinkle composition.

112. The composition of claim 106, wherein said hair care composition is capable of holding a hairstyle or for shaping hair.

113. The composition of claim 106, wherein said composition is in the for of a foundation or a lipstick.

114. A method for making up or caring for keratin materials comprising:
providing a cosmetic or dermatological composition comprising, in a physiologically acceptable medium, at least one copolymer having at least two ends, wherein at least one end is functionalized with an iodine atom, said copolymer comprising at least two different monomers, wherein one of the at least two different monomers is chosen from at least one soluble monomer having a homopolymer solubility of at least 3% by weight in isododecane at 25° C. and 1 atm, and the other of the at least two different monomers is chosen from at least one insoluble monomer having a homopolymer solubility of less than 3% by weight in isododecane at 25° C. and 1 atm; and
applying said composition to keratin materials.

115. The method of claim 114, wherein said keratin materials are chosen from body or facial skin, lips, nails, hair, and eyelashes.

116. The method of claim 114, wherein said composition is in the form of a cosmetic foundation or lipstick.
